(19) United States
(12) Patent Application Publication
(10) Pub. No.: US 2014/0171861 A1
(43) Pub. Date: Jun. 19, 2014

(54) SYRINGE ASSEMBLY

(71) Applicant: Allergan, Inc., Irvine, CA (US)

(72) Inventor: Dimitrios Stroumpoulis, Goleta, CA (US)

(73) Assignee: Allergan, Inc., Irvine, CA (US)

(21) Appl. No.: 14/135,184

(22) Filed: Dec. 19, 2013

Related U.S. Application Data
(60) Provisional application No. 61/739,549, filed on Dec. 19, 2012.

(51) Int. Cl. A61M 5/24; A61M 5/31

(52) U.S. Cl. CPC A61M 5/2448; A61M 5/3134
USPC 604/85

(57) ABSTRACT

An insert useful with a dermal filler syringe is provided for facilitating mixing of active agents with dermal fillers being injected into skin.

This application claims priority to and the benefit of U.S. Provisional Patent Application No. 61/739,549, filed Dec. 19, 2012, the entire disclosure of which is incorporated herein by this reference.

The present invention generally relates to syringes for introducing medicaments and more specifically relates to syringes for introducing dermal fillers for enhancement or tissue reconstruction purposes.

### BACKGROUND

Additives can provide a number of benefit when added to soft tissue fillers, for example, hyaluronic acid-based dermal fillers such as those marketed under the tradename Juvederm®, available from Allergan, Inc. Additives, such as anesthetics for example, when combined with dermal fillers or when introduced during a dermal filler injection procedure, may substantially improve patient comfort. Other additive, such as vitamins and antioxidants, may provide other clinical benefits when introduced into skin during the injection procedure. Certain commercial dermal fillers, such as Juvederm XC, for example, include anesthetics, specifically lidocaine, as an ingredient in the product.

Conventionally, additives that are not already included as an ingredient of a dermal filler product, are sometimes introduced prior to or after dermal filler injection, using a separate syringe. In certain cases the filler and the additive cannot be mixed until a few minutes prior to the injection, for example, in cases where mixing of the two fluid will cause instability or degradation of one or the other.

There is a need for methods and devices that facilitate mixing of a dermal filler with an additive immediately prior to injection therefore into skin.

### SUMMARY

Accordingly, assemblies, devices and methods are provided for mixing additive with dermal filler compositions prior to or concurrently with injection of the dermal filler/additive mixture into skin.

The present invention generally relates to a syringe assembly that is designed to mix and dispense a first injectable fluid and an additive. The first injectable fluid is stored in a syringe and the other is stored or immobilized in a syringe accessory hereinafter referred to as an insert, connectable to the syringe.

In one aspect of the invention, a syringe assembly is provided. The assembly generally comprises a syringe having a barrel for containing a fluid, for example a dermal filler, to be introduced into a body, a plunger slidably disposed in the barrel, and a distal portion defining a fluid flow path. The syringe further comprises a luer connector disposed at the distal portion of the syringe. The assembly further comprises an insert, coupled to the luer connector, comprising an additive containment member in line with the fluid flow path.

In one embodiment, the insert is removably coupled to the luer connector. For example, the insert may comprise a housing having a portion, for example, a proximal portion, that is couplable with the luer connector and a distal portion couplable with a needle or cannula hub. In use, the insert is coupled to the syringe such that the additive containment member is located in line with a flow of dermal filler being ejected from the syringe, for example, by means of the plunger. As the dermal filler material passes by the containment member, additive within the containment member becomes drawn into the dermal filler and is combined therewith. Mixing of dermal filler and additive can be enhanced by provision of structure, for example, a mixing chamber, as a part of the assembly, for example, in the insert distal to, downstream, or adjacent the containment member.

In some embodiments, the additive containment member comprises a matrix disposed in the housing and containing the additive to be introduced into the body in conjunction with the fluid, for example, dermal filler, contained in the barrel. In one embodiment, the matrix is a cylindrical body disposed along an inner wall of the insert. The matrix may be saturated with the additive, or the additive may otherwise be contained in or by the matrix. The matrix may be a fiber material, a sponge-like material or other suitable material that will temporarily contain an additive, but which will release the additive when a flow of the dermal filler is passed through or in contact with the matrix.

The additive may be any additive that will provide beneficial property when combined with a dermal filler, and may be provided in any suitable form. For example, the additive may be one or more of a anesthetic, a vasoconstrictor, an antioxidant, an enzymatic degradation inhibitor, and an antibiotic.

### BRIEF DESCRIPTION OF THE DRAWINGS

The present invention may be more thoroughly appreciated and/or the advantages and features thereof better understood, with reference to the following Detailed Description, when considered in conjunction with the accompanying Drawings of which:

FIG. 1 is a cross-sectional, exploded view of a syringe assembly in accordance with an embodiment of the invention;

FIG. 2 is a cross-sectional view of an insert of the syringe assembly shown in FIG. 1; and

FIG. 3 is a cross-sectional view of a portion of the syringe assembly including insert shown in FIGS. 1 and 2, as the assembly is being used to inject a dermal filler and additive.

### DETAILED DESCRIPTION

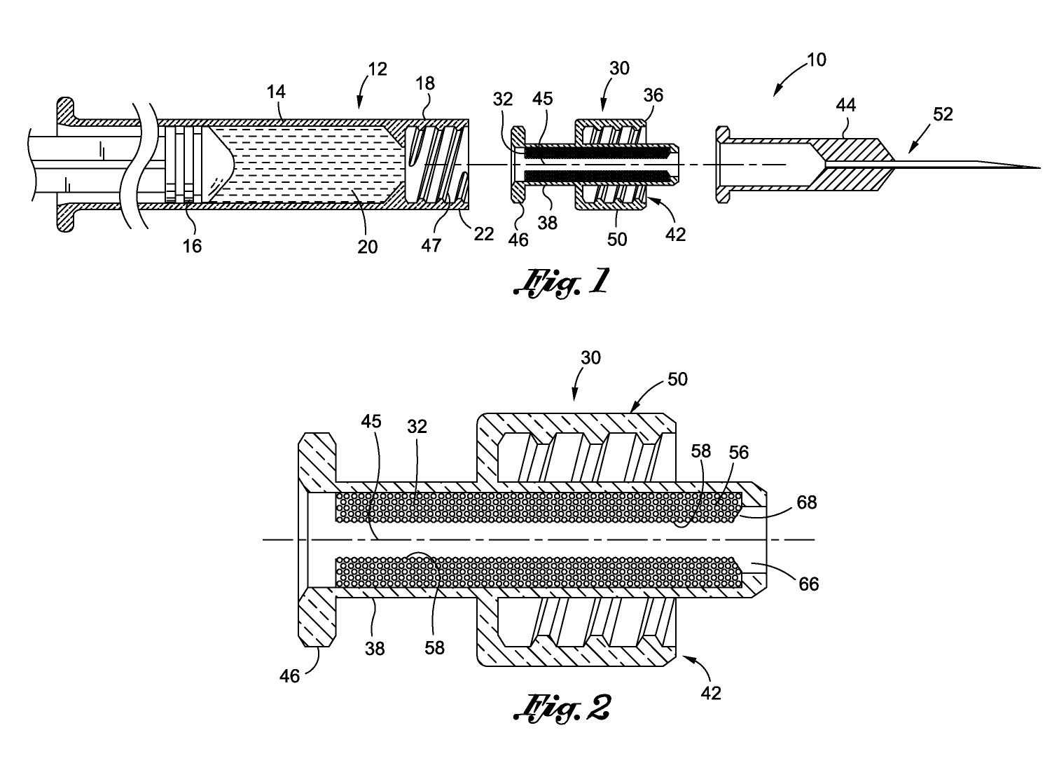

Turning now to FIG. 1, a dermal filler syringe assembly is shown generally at 10. The assembly 10 generally comprises a syringe 12 having a barrel 14 for containing a fluid, for example a dermal filler, to be introduced into a body, for example, skin, a plunger 16 slidably disposed in the barrel 14, and a distal portion 18 defining a fluid flow path 20, and a luer connector 22 disposed at the distal portion 18 of the syringe 12. The assembly 10 further comprises an insert 30, coupled, for example, removably coupled, to the luer connector 22. The insert 30 comprises an additive containment member 32 in line with the fluid flow path 20.

In the embodiment shown, the insert 30 comprises a housing 36 including a proximal portion 38 couplable with the luer connector 22 and a distal portion 42 couplable with a needle or cannula hub 44. More specifically, insert 30 includes additive containment lumen 45 and flange 46 that engages threads of luer connector 22. Housing 36 further includes threaded portion 50 circumscribing at least a portion of additive containment lumen 45, as shown. Threaded portion 50 is couplable to a hub 44 of a standard needle/cannula assembly 52. The coupling arrangement between syringe 12, needle/cannula assembly and insert 30 of this exemplary embodiment may be more clearly understood with reference to FIG. 3.

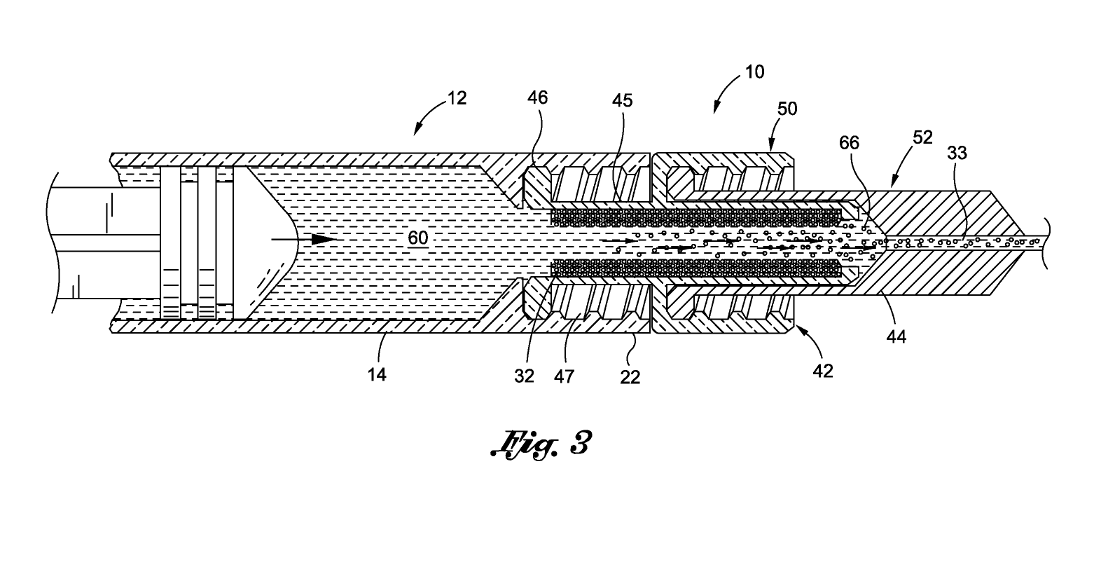

Turning now to FIGS. 2 and 3, the additive containment member 32 may comprise any suitable element or material that effectively holds the additive to be mixed with the dermal filler fluid. For example, the additive containment member 32 may comprise a matrix 56 disposed in the housing 36 and containing an additive 58 to be introduced into the body in conjunction with a fluid contained in the barrel 14. The matrix may be an absorbent material, for example, cotton or synthetic fiber material, or a porous sponge-like material such as a polyurethane foam, which is saturated with the additive. The matrix 56 may be in the form of a cylindrical, annular body disposed along an inner wall of the insert 30, as shown.

In one embodiment, the matrix 56 comprises an erodible or dissolvable polymer combined with the additive. When dermal filler 60 passes through passageway defined by insert 30, polymer/additive 58 is eroded from the matrix 46 and mixed into the dermal filler. To facilitate combining of the additive with the dermal filler, the insert 30 may include a mixing chamber 66, for example, located distal to matrix 46. Matrix 46 may be shaped to define portion of mixing chamber 66. For example, matrix 46 may include tapered distal annular surface 68, shown most clearly in FIG. 2.

Referring now briefly to FIG. 3, the assembly 10 of the invention operates generally as follows. When fluid, for example, dermal filler 60, is passed through the syringe barrel 14 by means of the plunger 16, additive 58 contained within the additive containment member 32 will be taken up in the flowing derma filler 60 and a mixture 33 of dermal filler/additive will be ejected from syringe 12.

Release of the additive and incorporation into the filler stream (see arrows, FIG. 3) may be accomplished by the combined action of convective mass transport, in that the viscous flow of the filler at the interface with a thin layer of the additive solution immobilized on the surface of the matrix 56, results in shear stress transfer and flow. Also, the additive may be diffused into the dermal filler by diffusive release, in that the additive in solution form is removed from the surface of the matrix, and is rapidly replaced by fresh additive solution diffusing out from the bulk of the saturated matrix. At the same time, the soft matrix may be compressed as the overall volume of the fluid it holds is reduced and as pressure is exerted by the filler. The matrix may be made of a material that is substantially impermeable to the filler, for example, due to its small porosity. The concentric flow of the additive solution relative to the filler is sustained by the rapid interchange between convective mass transport and diffusive release from the bulk of the matrix.

The additive may be any one of an anesthetic, a vasoconstrictor, an antioxidant, an enzymatic degradation inhibitor, and an antibiotic. The additive may be in any suitable form that allows release of the additive into the dermal filler flow.

Although the injectable fluid has been primarily described herein as an injectable dermal filler, it should be appreciated that the assembly 10 can be configured to be effective to introduce any two or more injectable fluids. For example, the assembly 10 can be used to introduce a first injectable fluid and the additive, which may be referred to hereinafter as a second injectable fluid.

The injectable fluids (e.g., the first injectable fluid or the second injectable fluid) are generally any biocompatible materials that when combined provide a benefit to the patient. These biocompatible materials include, but are not limited to, dermal fillers, for example, hyaluronic acid-based dermal fillers (e.g., Juvederm™ XC (Allergan, Irvine, Calif.)), hydrogels (i.e., superabsorbent natural or synthetic polymers), encapsulated and/or cross-linked biomaterials, silicones, glycosaminoglycans (e.g., chondroitin sulfate, dermatin sulfate, dermatin, dermatin sulfate, heparin sulfate, hyaluronic acid, o-sulfated hyaluronic acid), polysaccharides (e.g., chitosan, starch, glycogen, cellulose), collagen, elastin, local anesthetics (e.g., Benzocaine, Chloroprocaine, Cyclomethycaine, Dimethocaine/Larocaine, Propoxycaine, Procaine/Novocaine, Proparacaine, Tetracaine/Amethocaine, Amino amides, Articaine, Bupivacaine, Carticaine, Cinchocaine/Dibucaine, Etidocaine, Levobupivacaine, Lidocaine/Lignocaine, Mepivacaine, Piperocaine, Prilocalne, Ropivacaine, Trimecaine), drugs, bioactive agents, antioxidants, enzyme inhibitors (e.g., anti-hyaluronidase), vitamins, minerals, water, saline, light curable or light activated materials, vaccines, and pH curable or pH activated materials. Other biocompatible materials not mentioned above are also considered within the scope of the present invention.

The additive, or second injectable fluid, may comprise, for example, any bioactive agent which provides a benefit to the patient, or one which facilities delivery of the first injectable fluid (e.g., to reduce extrusion force and/or viscosity). Additional bioactive agents may include anti-proliferatives including, but not limited to, macrolide antibiotics including FKBP-12 binding compounds, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, and anti-inflammatories. Drugs can also refer to bioactive agents including anti-proliferative compounds, cytostatic compounds, anti-inflammatory compounds, anti-fungal agents, steroids, chemotherapeutic agents, analgesics, antibiotics, protease inhibitors, statins, nucleic acids, polypeptides, growth factors and delivery vectors including recombinant micro-organisms, liposomes, and the like. Combinations of additional bioactive agents are also within the scope of the present invention.

Unless otherwise indicated or otherwise clearly contradicted by context, combinations of the above-described elements in all possible variations thereof are contemplated to be included within the scope of the invention.

What is claimed is:

1. A syringe assembly comprising:
a syringe having a barrel for containing a fluid to be introduced into a body, a plunger slidably disposed in the barrel, and a distal portion defining a fluid flow path;
a luer connector disposed at the distal portion of the syringe;
an insert, coupled to the luer connector, comprising an additive containment member in line with the fluid flow path.

2. The assembly of claim 1 wherein the insert is removably coupled to the luer connector.

3. The assembly of claim 1 wherein the insert further comprises a housing couplable with the luer connector.

4. The assembly of claim 1 wherein the housing comprises a proximal portion couplable with the luer connector and a distal portion couplable with a needle or cannula hub.

5. The assembly of claim 3 wherein the additive containment member comprises a matrix disposed in the housing and containing an additive to be introduced into the body in conjunction with a fluid contained in the barrel.

6. The assembly of claim 5 wherein the matrix is saturated with the additive.

7. The assembly of claim 5 wherein the matrix is a fiber material.

8. The assembly of claim 5 wherein the matrix is a cylindrical body disposed along an inner wall of the insert.

9. The assembly of claim 1 wherein the insert includes a mixing chamber.

10. The assembly of claim 5 wherein the insert includes a mixing chamber distal to the matrix.

11. An insert useful with a dermal filler syringe having a barrel for containing a fluid to be introduced into a body, a plunger slidably disposed in the barrel, and a distal portion including a luer connector and defining a fluid flow path, the insert comprising:
a housing including a proximal portion configured to be couplable with the luer connector of said syringe and a distal portion configured to be couplable with a needle or cannula hub; and
an additive containment member disposed in the housing and in line with the fluid flow path, the additive containment member containing an additive to be introduced into the body in conjunction with a dermal filler contained in the barrel of said syringe when the insert is coupled to said syringe.

12. The insert of claim 10 wherein the additive containment member comprises a matrix disposed in the housing.

13. The insert of claim 10 wherein the additive containment member comprises a matrix disposed in the housing, the matrix being saturated with the additive.

14. The insert of claim 11 wherein the matrix is a fiber material.

15. The insert of claim 11 wherein the matrix is a cylindrical body disposed along an inner wall of the insert.

16. The insert of claim 10 wherein the housing defines a mixing chamber.

17. The insert of claim 11 wherein the housing defines a mixing chamber distal to the matrix.
